(19) United States
(12) Patent Application Publication
(10) Pub. No.: US 2016/0081666 A1
(43) Pub. Date: Mar. 24, 2016

(54) ULTRASONIC DIAGNOSTIC DEVICE

(71) Applicant: HITACHI ALOKA MEDICAL, LTD., Tokyo (JP)

(72) Inventors: Takashi Deguchi, Mitaka-shi (JP); Jing-Wen Tsao, Mitaka-shi (JP); Mitsuru Ueda, Mitaka-shi (JP)

(73) Assignee: HITACHI ALOKA MEDICAL, LTD., Mitaka-shi, Tokyo (JP)

(21) Appl. No.: 14/781,631

(22) PCT Filed: Apr. 21, 2014
(86) PCT No.: PCT/JP2014/061207

§ 371 Date: Oct. 1, 2015

(30) Foreign Application Priority Data
Apr. 22, 2013 (JP) 2013-089818

(51) Int. Cl. A61B 8/00; A61B 8/08

(52) U.S. Cl. CPC A61B 8/54; A61B 8/0841; A61B 8/5207; A61B 8/5246; A61B 8/463; A61B 8/4494; A61B 8/467

(57) ABSTRACT

A graphic image displayed on a tomographic image includes a puncture guide. The puncture guide has a main guide line and a sub guide line. The main guide line represents a reference puncture route based on a puncture angle, and the sub guide line represents the edge of a composite area defined by a plurality of scans. A deflection angle (θ1) is determined according to the designation of a puncture angle (φ1), and the puncture guide is formed on the basis of these angles.

### TECHNICAL FIELD

The present invention relates to an ultrasound diagnostic apparatus, and in particular to a technique for assisting freehand puncturing.

### BACKGROUND ART

A puncture technique or a puncture maneuver (hereinafter simply referred to as “puncture”) is a technique for inserting a puncture needle into a body through a body surface, in order to inject medicine, sample a tissue, or the like. The puncturing may also be executed for cauterizing a tumor or the like. For the puncturing, ultrasound diagnostic is utilized in order to check a positional relationship between a tip of the puncture needle and a target tissue. Specifically, the puncture needle is inserted while the user checks a puncture needle image appearing on a tomographic image. As the method of puncturing, there are known a “mechanical guide method” in which a puncture adapter having a needle groove is mounted on a probe and the puncture needle is guided by the puncture adapter, and a “freehand method” in which the puncture needle is held by hand without the use of such equipment and the puncturing is freely executed. In either method, a direction of movement of the puncture needle is determined such that a puncture route is in a beam scan plane.

Electronic linear scanning includes non-deflection scanning and deflection scanning. In the non-deflection scanning, the ultrasound beam is not deflected. That is, the ultrasound beam is formed with a beam deflection angle of 0 degrees (in a direction orthogonal to a transmission and reception plane of a transducer), and such a vertical ultrasound beam is electronically scanned. In such non-deflection scanning, a rectangular beam scan area is formed for each scan, and a tomographic image corresponding thereto (non-deflection scan image) is formed.

On the other hand, in the deflection scanning, the ultrasound beam is tilted at a desired deflection angle, and the tilted ultrasound beam is electronically scanned. In the case of the deflection scanning, normally, a parallelogram-shaped beam scan plane is formed for each scan, and a tomographic image corresponding thereto (deflection scan image) is formed. In relation to this scanning, a special compound method is known for improving the image quality. In this method, for example, frame data formed by non-deflection scanning, frame data formed by deflection scanning with a positive deflection angle, and frame data formed by deflection scanning with a negative deflection angle are combined to produce combined frame data, and a tomographic image is formed based on the combined frame data. In this case, normally, of the individual deflection scan frame data, a triangular portion which extends beyond the non-deflection frame data is cut out, and a tomographic image having a rectangular shape similar to the tomographic image for the non-deflection scanning is formed. The tomographic image comprises a central sub area in which the non-deflection scan area and two deflection scan areas are overlapped, and a right-side sub area and a left-side sub area in each of which the non-deflection scan area and one deflection scan area are overlapped. In a convex scan or the like, in which the ultrasound beam is scanned in a fan shape, also, the spatial compound method may be applied in some cases.

The appearance of the puncture needle image on the tomographic image significantly changes depending on an intersection angle between an axial direction of the puncture needle (puncture direction) and the ultrasound beam. In general, an intensity of an echo from the puncture needle is maximized when the intersection angle is a right angle, and the puncture needle image appears most significantly on the tomographic image in such a case. Therefore, when puncturing is to be executed, it is desirable to execute the deflection scanning after setting the beam deflection angle such that the beam direction is orthogonal to the puncture route. On the other hand, in order to form an image of tissue, or to form a rectangular tomographic image as in the normal case, it is desirable to execute the non-deflection scanning. From such a viewpoint, during puncture, the non-deflection scanning and the deflection scanning are alternately repeated, and tomographic images based on the combined frame data are sequentially displayed.

Patent Document 1 discloses an ultrasound diagnostic apparatus which is used for the puncturing. In the ultrasound diagnostic apparatus, a puncture adapter is mounted on a probe, and the puncture angle is specified by the type of the puncture adapter. The deflection angle of the ultrasound beam is set to be orthogonal to the puncture route based on the puncture angle. In other words, the ultrasound beam in a tilted state is electronically scanned. With such a configuration, a tomographic image including a tissue image and a puncture needle image is displayed on a screen. A guide line representing the puncture route is also included in the display on the screen. However, in the ultrasound diagnostic apparatus of Patent Document 1, during the puncturing, only the deflection scan image is displayed. Patent Document 1 does not disclose the use of the combined image based on the spatial compound method, and consequently does not describe a unique problem associated with the display of the puncture needle image on the combined image and a structure for solving the problem.

Patent Document 2 discloses, in FIG. 5 an ultrasound diagnostic apparatus which combines a non-deflection scan image and a deflection scan image, and displays a combined image. The former is for clearly displaying the tissue, and the latter is for clearly displaying the puncture needle. With the combined image, both the tissue and the puncture needle can be clearly displayed. However, Patent Document 2 does not disclose a unique problem associated with differing shapes of the non-deflection scan image (non-deflection scan area) and the deflection scan image (deflection scan area), and a structure for handling the problem. More specifically, in Patent Document 2, the combined image is a rectangular image, and includes an overlap sub area formed by both images and a non-overlap subarea formed by only the non-deflection scan image. In the non-overlap sub area, normally, the puncture needle is not clearly displayed. It is therefore desirable that, prior to the puncturing, the target tissue be positioned within the overlap sub area and the puncture needle be set to not reach the non-overlap sub area. However, a boundary between the overlap sub area and the non-overlap sub area is not necessarily clear on the combined image. Patent Document 2 does not describe a structure for explicitly showing the boundary.

In the structure shown in FIG. 5 of Patent Document 2, the puncture adapter is used (mechanical guide method is employed), and the puncture angle is determined before the puncturing. Thus, a constant puncture route is always formed. In this case, because the deflection angle is always a constant, the position of the boundary does not vary, and the necessity for having attention of the user on the position of the boundary for each inspection is low. Alternatively, the puncture angle (that is, the deflection angle) may be determined such that the overlap sub area is dominant in the combined image. In this case, the target tissue would be positioned within the overlap sub area in most situations, and thus the possibility of having the above-described problem is low.

On the contrary, in the case of the freehand method, in particular, in the freehand method in which the deflection angle can be freely selected, the position of the boundary may change for every inspection, and in the first place, it is difficult to recognize the deflection angle on the screen. Even if a puncture guide line is displayed, only a rough estimate of the puncture direction can be obtained, and it is difficult to identify an edge of the overlap sub area (the above-described boundary). Therefore, it is difficult to recognize on the image whether or not the target tissue (or a point where the tip of the puncture needle reaches) is positioned within the overlap sub area. In the case of the freehand method, it is necessary to hold the probe with one hand and hold the puncture needle at the same time with the other hand, and it is difficult to designate or select the deflection angle after the puncturing is started. The deflection angle must be designated before the puncturing.

Patent Document 3 shows an image combining system based on the spatial compound method. The system appears to assume the freehand method. In this system, in order to clearly form the images of both the tissue and the puncture needle, a plurality of tomographic images having different deflection angles are combined. Patent Document 3 shows, in FIG. 8, a graphic representing a trapezoidal sub area in the combined image. The trapezoidal sub area may be understood to be an area where the non-deflection scan image and the deflection scan image overlap each other, and a periphery thereof is graphically represented. However, in the structure of FIG. 8 of Patent Document 3, there is no element corresponding to the puncture guide line. Because the direction of movement of the puncture needle appears to be not orthogonal to the slanted side of the trapezoidal sub area in FIG. 8, Patent Document 3 does not recognize establishment of the orthogonal relationship.

### RELATED ART REFERENCES

### Patent Documents [Patent Document 1] JP H9-28708 A [Patent Document 2] JP 2006-320378 A [Patent Document 3] US Patent Application Publication No. 2011/0249878 A

### DISCLOSURE OF INVENTION

### Technical Problem

An advantage of the present invention is that an inspector is enabled to check a reference puncture route which changes according to a beam deflection angle prior to the puncturing, and to check a boundary of the puncture needle image forming sub area which changes according to the beam deflection angle. Another advantage of the present invention is that the inspector is enabled to easily check that the target tissue is positioned within the puncture needle image forming sub area prior to the puncturing.

### Solution to Problem

According to one aspect of the present invention, there is provided an ultrasound diagnostic apparatus comprising: a deflection angle determination unit that determines a beam deflection angle corresponding to a planned puncture angle; a scan controller that controls a first beam scan for forming a tissue image, and a second beam scan for forming a puncture needle image according to the beam deflection angle; a graphic image formation unit that forms a graphic image having a puncture guide; a combining unit that combines first frame data obtained by the first beam scan and second frame data obtained by the second beam scan, to produce combined frame data; and a display processor that combines an ultrasound image based on the combined frame data and the graphic image, to produce a display image, wherein the puncture guide includes: a main guide line that represents a reference puncture route determined from the planned puncture angle; and a sub guide line that intersects the main guide line, that is shorter than the main guide line, and that represents an edge of an area of the second beam scan.

According to the above-described configuration, a graphic image is displayed along with the ultrasound image, and the graphic image includes a puncture guide. The puncture guide is for assisting the puncturing, and includes a main guide line and a sub guide line. With the main guide line, a reference puncture route can be visually recognized. The reference puncture route functions as a rough estimate for obtaining a clear puncture needle image. Even if the puncture needle deviates from the reference puncture route, the object of puncture can be achieved so long as the actual puncture route for the target tissue is not deviated from. When an angle of deviation between the reference puncture route and the actual direction of movement of the puncture needle becomes large, the clearness of the puncture needle image would be degraded. It is therefore desirable to execute the puncturing such that the actual puncture route is as close to, or as parallel to, the reference puncture route as possible. When the reference puncture route is determined to just pass the puncture target coordinate, it is desirable to execute the puncturing such that the actual puncture route is as close to the reference puncture route as possible. Desirably, the reference puncture route is determined by a reference point and the planned puncture angle. The reference point is, for example, a point at a top right corner or a top left corner of the ultrasound image, corresponding to one end or the other end of the probe (or one end or the other end of an array transducer). Alternatively, the reference point may be set not as a fixed point, but variable depending on the actual puncture position.

The sub guideline represents an edge of the second beam scan area. In other words, with the sub guide line, a sub area which can display a clear puncture needle image (an area in which the second beam scan is executed; the overlap sub area) can be visually easily identified. Specifically, it is desirable that the planned puncture angle is determined and a position and an orientation of the probe are determined such that the target tissue or a puncture target coordinate to be reached by the puncture needle tip is included in the sub area. According to such a setting, it is possible to continue to always clearly display the puncture needle image (in particular, the tip thereof) during the puncture process. That is, a problem in that the puncture needle tip is moved out of the sub area and the image of the puncture needle tip is suddenly not formed or becomes unclear can be resolved.

In the above-described configuration, when the planned puncture angle is determined by a manual input or by an automatic determination, the beam deflection angle in the second beam scan is accordingly automatically determined, and the puncture guide is accordingly automatically produced. In this case, the beam deflection angle is determined such that the ultrasound beam intersects the reference puncture route, preferably, at an intersection angle close to orthogonal angle, and more preferably, orthogonally. In addition, the angle and position of the main guide line, and the angle and position of the sub guide line, are determined. When the planned puncture angle is changed, the angle and position of the main guide line and the angle and position of the sub guide line change accordingly. Preferably, when the planned puncture angle (angle of the reference puncture route with respect to the horizontal line) is reduced such that the reference puncture route is moved closer to the horizontal line, the sub guide line is slid and moved in a deeper direction (direction away from the puncture start position) on the main guide line. In this process, the display form of the sub guide line may be changed. For example, a length of the sub guide line may be increased. The planned puncture angle and the beam deflection angle are in a correspondence relationship. That is, when one is specified, the other would be defined. Therefore, in place of designating the former, the latter may be designated. Such a form may be conceptualized as an indirect designation of the planned puncture angle.

In the first beam scan, preferably, a non-deflection beam is scanned, but alternatively, a deflection beam may be scanned. Alternatively, in the first beam scan, scan of the non-deflection beam and scan of the deflection beam (for example, beam scan in a symmetry relationship in the left-right direction with the second beam scan) may be sequentially executed. The above-described structure is particularly effective in the freehand puncturing, but the structure may alternatively be used for other punctures.

Preferably, the sub guide line is orthogonal to the main guide line, and a combination of the main guide line and the sub guide line forms a cross shape or a T shape. According to this configuration, the relationship of the reference puncture route and the deflection beam orthogonal thereto can be visually instantaneously recognized. When a cross shape is employed, the main guide line can be displayed over the entirety of the display frame, which results in easier recognition of the reference puncture route. When the T shape is employed, the edge of the second beam scan area can be more easily recognized.

Preferably, coordinates of both ends of the sub guide line represent a lower limit and an upper limit of a recommended puncture angle range determined with reference to the reference puncture route. According to such a configuration, in the insertion process of the puncture needle, a degree of appropriateness of the insertion angle and the insertion position of the puncture needle can be recognized, with a width of the sub guide line serving as a rough estimate. Alternatively, a marker representing a triangular region connecting a starting point of the reference puncture route and both ends of the sub guide line may be displayed. Alternatively, a marker representing the recommended puncture angle range may be displayed partway on the reference puncture route.

Preferably, a position of the sub guide line is moved toward a deeper position along the main guide line and a line length of the sub guide line is increased as the planned puncture angle is reduced and the reference puncture route becomes closer to a horizontal line. Preferably, the upper limit and the lower limit of the recommended puncture angle range are determined by an addition and a subtraction of a predetermined angle to and from the planned puncture angle.

Preferably, the ultrasound diagnostic apparatus further comprises an emphasis processor that applies an emphasis processing to emphasize an image of the puncture needle on the second frame data, wherein the combining unit combines the second frame data after the emphasis processing to the first frame data. Preferably, the emphasis processing includes an edge emphasis processing.

Preferably, the ultrasound diagnostic apparatus further comprises a probe that has a transmission and reception end in which an array transducer formed from a plurality of transducer elements that transmit and receive ultrasound is built in, and that is is in contacted with a surface of a living body, and a mode selector that selects a display mode from among a first display mode adapted for one side procedure in which puncturing is executed on one side of the transmission and reception end, and a second display mode adapted for the other side procedure in which the puncturing is executed on the other side of the transmission and reception end, wherein when the first display mode is selected, the scan controller sets a positive angle as the beam deflection angle, and a forward tilted puncture guide having a main guide line tilted from an upper part on the one side toward a lower part on the other side is displayed as the puncture guide, and when the second display mode is selected, the scan controller sets a negative angle as the beam deflection angle, and a reverse tilted puncture guide having a main guide line tilted from an upper part on the other side toward a lower part on the one side is displayed as the puncture guide. According to such a configuration, a display corresponding to both the one side procedure and the other side procedure can be provided. Therefore, the procedure may be selected according to the dominant arm, the state of the target tissue, or the like.

Preferably, the display processor inverts the ultrasound image and the graphic image in the left-right direction when an inversion command is input. For example, there may be cases where the tomographic image displayed on the screen and the actual space do not match each other depending on the orientation of the probe, but with the above-described configuration, such mismatch may be resolved.

Preferably, the ultrasound diagnostic apparatus further comprises an input unit that allows variation of the planned puncture angle, wherein a real-time tomographic image is displayed as the ultrasound image, and the beam deflection angle in the second beam scan and a display form of the puncture guide are updated in real time according to a change of the planned puncture angle. Preferably, the ultrasound diagnostic apparatus is an apparatus used when freehand puncturing is executed without the use of a puncture adapter that mechanically guides a puncture needle. Alternatively, a groove or a recess with which the puncture needle is in contact may be formed on one end and the other end of the probe. According to such a configuration, the orientation of the puncture needle can be stabilized during insertion. In addition, a problem in that the puncture needle deviates from the scanning plane from the beginning can be avoided.

According to another aspect of the present invention, there is provided a method of forming an image, comprising: a step of forming an ultrasound image based on combined frame data; a step of forming a graphic image including a puncture guide; and a step of combining the ultrasound image and the graphic image to form a display image, wherein the combined frame data is produced by combining first frame data obtained by a first beam scan for forming a tissue image and second frame data obtained by a second beam scan for forming a puncture needle image corresponding to a beam deflection angle corresponding to a planned puncture angle, and the puncture guide includes: a main guide line that represents a reference puncture route determined from the planned puncture angle; and a sub guide line that is orthogonal to the main guide line and that represents an edge of an area of the second beam scan. This method is executed on an ultrasound diagnostic apparatus.

### BRIEF DESCRIPTION OF DRAWINGS

FIG. 1 is a block diagram showing an ultrasound diagnostic apparatus according to a preferred embodiment of the present invention.

FIG. 2 is a diagram showing an example operation of the ultrasound apparatus shown in FIG. 1.

FIG. 3 is a diagram showing a first example of a puncture guide.

FIG. 4 is a diagram for explaining significance of a sub guide line in a puncture guide.

FIG. 5 is a diagram showing a display image immediately after puncture is started.

FIG. 6 is a diagram showing a display image representing a puncture needle which has reached a target tissue.

FIG. 7 is a diagram showing a state where a target tissue deviates from a puncture needle image area.

FIG. 8 is a diagram showing a state where a puncture needle image deviates from a reference puncture route.

FIG. 9 is a diagram showing a state where a tip of a puncture needle image deviates from a recommended angle range.

FIG. 10 is a diagram for explaining a change of a puncture guide due to variation of a puncture angle.

FIG. 11 is a diagram showing a puncture guide in a second display mode.

FIG. 12 is a diagram showing a puncture guide in an inverted display mode.

FIG. 13 is a diagram showing a second example of a puncture guide.

FIG. 14 is a diagram showing a third example of a puncture guide.

FIG. 15 is a diagram showing a fourth example of a puncture guide.

FIG. 16 is a diagram showing a fifth example of a puncture guide.

FIG. 17 is a diagram showing a sixth example of a puncture guide.

FIG. 18 is a flowchart for explaining a procedure for executing freehand puncturing.

FIG. 19 is a diagram showing convex scanning.

FIG. 20 is a diagram showing a deflection scan in a convex probe.

FIG. 21 is a diagram showing a puncture guide displayed on an image formed using a convex probe.

### EMBODIMENT

A preferred embodiment of the present invention will now be described with reference to the drawings.

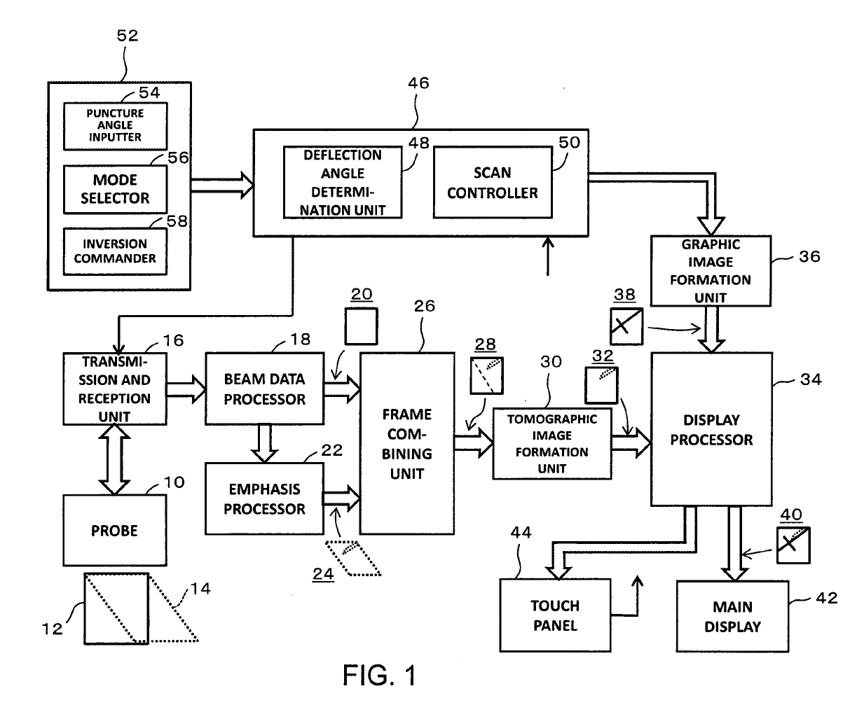

FIG. 1 shows an ultrasound diagnostic apparatus according to a preferred embodiment of the present invention. FIG. 1 is a block diagram showing an overall structure of the ultrasound diagnostic apparatus. The ultrasound diagnostic apparatus shown in FIG. 1 is an apparatus which is provided in a medical institute such as a hospital, and which forms an ultrasound image representing the inside of a living body by transmission and reception of ultrasound to and from the living body. The ultrasound diagnostic apparatus according the present embodiment particularly has an operation mode adapted for freehand puncturing.

In the example configuration of FIG. 1, a probe 10 is a linear probe. The probe 10 has a transmission and reception end (lower end) which is in contact with a surface of a living body, and an array transducer made of a plurality of transducer elements is provided in the transmission and reception end. The plurality of transducer elements are arranged in a straight line shape. An ultrasound beam is formed by the array transducer, and is electronically scanned. In the present embodiment, an electronic linear scanning method is applied. In the electronic scanning of the ultrasound beam, a deflection angle of the ultrasound beam can be set in a variable manner.

In the present embodiment, a first beam scan and a second beam scan are alternately executed. The first beam scan is an electronic scanning of an ultrasound beam for forming an image of a tissue, and in the present embodiment, a non-deflection scan plane 12 is formed. The non-deflection scan plane 12 is formed by setting 0 degrees as a deflection angle of the ultrasound beam and then linearly scanning the ultrasound beam. In the second beam scan, a deflection scan plane 14 is formed. The deflection scan plane 14 is formed by setting the ultrasound beam in a tilted state at a certain angle, and then linearly scanning the ultrasound beam while maintaining the deflection angle. The non-deflection scan plane 12 has a rectangular shape, and the deflection scan plane 14 has a parallelogram shape. The second beam scan is executed for forming a clear image of a puncture needle, and in the present embodiment, the deflection angle of the ultrasound beam is determined such that the ultrasound beam is orthogonal to a puncture route of the puncture needle. In other words, because the ultrasound would be transmitted at a right angle with respect to the puncture needle if the ultrasound beam is scanned under such a condition, a strong reflection wave can be obtained from the surface of the puncture needle. Alternatively, in order to form the image of the tissue, a deflection scan plane tilted in a direction opposite to the deflection scan plane 14 may be additionally formed.

As will be described later, first frame data corresponding to the non-deflection scan plane 12 is obtained by the first beam scan. The first frame data is formed from a plurality of beam data arranged in the electronic scanning direction. Each beam data is formed from a plurality of echo data arranged along a depth direction. On the other hand, second frame data corresponding to the deflection scan plane 14 is obtained by the second beam scan. The second frame data is also formed from a plurality of beam data arranged in the electronic scanning direction. Alternatively, the deflection scan plane 14 may be formed in place of the non-deflection scan plane 12, as the scan plane for forming the image of the tissue.

A transmission and reception unit 16 is a circuit that functions as a transmission beam former and a reception beam former. During transmission, the transmission and reception unit 16 supplies a plurality of transmission signals that are parallel to each other to a plurality of transducer elements forming a transmission opening. With such a process, a transmission beam is formed by the array transducer. During reception, when a reflected wave from the inside of the living body is received by the plurality of transducer elements forming a reception opening, a plurality of reception signals are output from the transducer elements to the transmission and reception unit 16 in parallel to each other. At the transmission and reception unit 16, a phased summation process is executed on the plurality of reception signals, to thereby form a reception signal after the phased summation, that is, the beam data. The beam data is output to a beam data processor 18. The transmission and reception processes are repeatedly executed while changing the positions of the transmission and reception openings, so that the first frame data and the second frame data as described above are obtained. In the present embodiment, the first frame data and the second frame data are alternately obtained.

The beam data processor 18 has known circuits such as a wave detector, a logarithmic compressor, or the like, and stepwise executes predetermined processes on input beam data. With this process, processed first frame data 20 and processed second frame data are alternately output from the beam data processor 18. The first frame data 20 is directly sent to a frame combining unit 26. The second frame data is sent to the frame combining unit 26 via an emphasis processor 22.

The emphasis processor 22 applies an emphasis processing to display frame data such that the puncture needle image is displayed in an emphasized manner. The emphasis processing includes an edge extraction processing. The emphasis processing may further include a threshold processing to extract a high brightness portion, a frame correlation processing, or the like. By applying these processings, it is possible to obtain frame data in which the portion of the puncture needle image is extracted, that is, only the portion of the puncture needle image is emphasized. Such frame data is shown in FIG. 1 as second frame data 24.

In the present embodiment, the first frame data 20 output from the beam data processor 18 is directly sent to the frame combining unit 26, but alternatively, the first frame data 20 may be output to the frame combining unit 26 after a predetermined process for clarifying the tissue image is applied to the first frame data 20.

The frame combining unit 26 is a circuit which combines the first frame data 20 and the second frame data 24, to produce combined frame data 28. The second frame data 24 has a portion that extends beyond the first frame data 20, and this portion is deleted in the present embodiment. In other words, the frame combining unit 26 has a shape-forming function. Alternatively, such a portion may be retained without deleting the portion, and an image thereof may be formed. In the present embodiment, the combined frame data 28 conceptualized as having a rectangular shape includes an overlap sub area in which the first frame data and the second frame data are overlapped, and a non-overlap sub area formed from only the first frame data. In the overlap sub area, the puncture needle image can be clearly displayed, but in the non-overlap sub area, the puncture needle image tends to be not clear.

A tomographic image formation unit 30 is formed from a digital scan converter (DSC) in the present embodiment, and is a circuit which forms a tomographic image (B mode tomographic image) 32 based on input combined frame data 28. The tomographic image formation unit 30 has a coordinate transformation function, an interpolation process function, or the like. In addition, the tomographic image formation unit 30 has a frame rate adjusting function or the like. The formed tomographic image 32 is sent to a display processor 34 in units of display frames.

On the other hand, a graphic image formation unit 36 produces a graphic image 38 including a puncture guide under the control of a controller 46. The graphic image formation unit 36 is formed as an image processing circuit, or as a function of a program executed by a CPU. As will be explained later, the puncture guide includes a plurality of graphic elements. More specifically, the puncture guide includes a main guide line and a sub guide line. The main guide line represents a reference puncture route, and the sub guide line represents an edge of the deflection scan plane 14 formed by the second beam scan, that is, an outer edge of a puncture needle image forming area.

The display processor 34 is a circuit which has an image combining function, a color process function, or the like, and which combines the graphic image 38 with the tomographic image 30, to form a display image 40. The display image 40 is displayed on a main display 42 formed from a flat panel display or the like. In addition, the display image 40 is displayed on a touch panel 44 including a sub display as necessary. On the touch panel 44, it is possible to designate the puncture angle, to select the mode, etc., as will be described later.

The controller 46 has a deflection angle determination unit 48 and a scan controller 50 in the present embodiment. The deflection angle determination unit 48 determines the deflection angle at the second beam scan based on a puncture angle designated by a user. The scan controller 50 controls the first beam scan and the second scan, and in the second beam scan, the ultrasound beam is deflected and scanned according to the deflection angle determined by the deflection angle determination unit 48. The controller 46 is formed from, for example, a CPU and an operation program. From the controller 46, information necessary for forming the graphic image, in particular, the information of the puncture angle and the deflection angle, is sent to the graphic image formation unit 36. The puncture angle and the deflection angle are in a correspondence relationship, and when one is specified, the other can be specified. Therefore, alternatively, a configuration may be employed in which only one of the information of the puncture angle and the information of the deflection angle is sent from the controller 46 to the graphic image formation unit 36.

An input unit 52 is connected to the controller 46. The input unit 52 is, for example, a manipulation panel. The input unit 52 includes a puncture angle inputter 54, a mode selector 56, and an inversion commander 58 in the present embodiment. The puncture angle inputter 54 is a unit for inputting a desired puncture angle by the user prior to the freehand puncturing. The user variably sets a suitable puncture angle in relation to the tissue, while referring to the puncture guide displayed on the tomographic image.

When the user varies the puncture angle, the format of the puncture guide (form and position) is changed in real time. In addition, when the puncture angle is varied, the deflection angle is also varied accordingly. That is, the scan condition in the second beam scan is changed. The user manipulates on the puncture angle inputter 54 to select a suitable puncture angle, and executes the freehand puncturing.

The mode selector 56 is a unit for allowing the user to select a display mode from either a first display mode or a second display mode. The first display mode is a display mode selected when puncturing is to be executed at a start end side of the electronic scanning at the probe 10 (when a one side maneuver is to be executed). The second display mode is a display mode selected when the puncturing is executed on a completion end side of the electronic scanning at the probe 10 (when the other side maneuver is to be executed). Two display modes are provided to allow selection of the side, with respect to the probe 10, where the puncturing is to be executed, for example, depending on the dominant arm. When the first display mode is selected, a positive angle is set as the deflection angle, for example, and a guide line which is tilted in the forward direction from the top right to the bottom left is displayed on the image. On the other hand, when the second display mode is selected, a negative angle is set as the deflection angle, and a main guide line which is tilted from the top left to the bottom right is displayed on the display screen.

The inversion commander 58 is a unit which gives a command to invert the tomographic image and the graphic image in the left-right direction as necessary. The inversion of the image may be executed at the display processor 34 or at the tomographic image formation unit 30 and the graphic image formation unit 36.

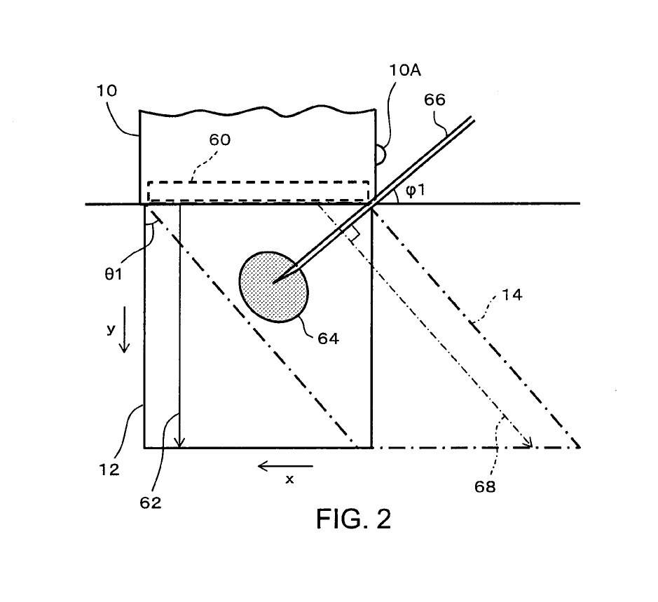

FIG. 2 schematically shows an example operation of the ultrasound diagnostic apparatus shown in FIG. 1. As described above, the probe 10 comprises a transmission and reception end, and an array transducer 60 is provided in the transmission and reception end. The array transducer 60 is formed from a plurality of transducer elements arranged in the electronic scanning direction. On one side of the transmission and reception end, a protrusion 10A is provided. The protrusion 10A is a marker indicating a start end side of the electronic scan, and is called a direction mark.

The ultrasound is transmitted and received while in a state where the transmission and reception plane of the probe 10 is in contact with the surface of the living body. As described above, the non-deflection scan plane 12 is formed by the first scan. Specifically, the non-deflection scan plane 12 is formed by electronically scanning, in an electronic scanning direction (x direction), an ultrasound beam 62 formed under a condition of the deflection angle of 0 degrees. However, in order to form an image of the tissue, a deflected ultrasound beam may be electronically scanned. Here, a y direction is a depth direction. The deflection scan plane 14 is formed by the second beam scan, and more specifically, is formed by electronically scanning, in the electronic scan direction, an ultrasound beam 68 deflected by a predetermined angle. In the present embodiment, a deflection angle of the ultrasound beam 68 is shown with an angle θ1. In FIG. 1, the angle θ1 is defined as an angle with respect to a vertical direction, and is a positive angle. In the present embodiment, the deflection angle θ1 is determined according to a puncture angle φ1 designated by the user prior to the freehand puncturing. The puncture angel φ1 shown in FIG. 2 is defined as an angle with respect to a horizontal line. The angle φ1 is a positive angle, and this does not change between the puncture on one side of the probe 10 and the puncture on the other side of the probe 10.

In the coordinate system of FIG. 2, φ1=θ1. That is, when the puncture angle φ1 is designated, the angle θ1 is determined. In the second display mode, if the puncturing is to be executed on the completion end side of the electronic scanning in the probe 10, φ1=−θ1. As will be described in detail below, the puncture angle φ1 is set while referring to the puncture guide, and the puncture needle 66 is then inserted into the living body in a manner to realize the puncture angle φ1. In FIG. 2, a state is shown in which a tip of the puncture needle 66 has reached a center of a target tissue 64.

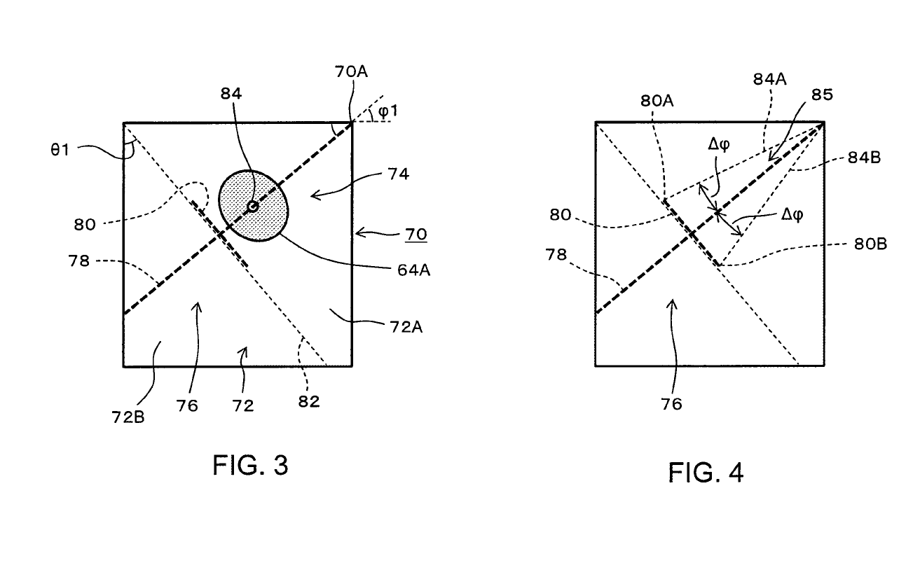

FIG. 3 shows a first example of the puncture guide. A display image 70 is an image produced by combining a tomographic image 72 and a graphic image 74. The tomographic image 72 includes a sub area 72A in which the first frame data and the second frame data are overlapped, and a sub area 72B where there is no such overlap. In the sub area 72A, the puncture needle image can be displayed relatively clearly. On the other hand, in the sub area 72B, even when the puncture needle enters the area, the puncture needle cannot be displayed too clearly. On the tomographic image 72, an edge (boundary) 82 of the sub area 72A may be recognized as a stripe, but in general, it is difficult to clearly identify the edge 82 on the image.

The graphic image 74 includes a puncture guide 76 in the present embodiment. The puncture guide 76 has a cross-like form in the first example shown in FIG. 3. Specifically, the puncture guide 76 includes a main guide line 78 and a sub guide line 80. The main guide line 78 is a line representing a reference puncture route, is tilted from a top right corner point 70A in the display image 70 toward the bottom left, and is drawn over the entirety of the display image 70. The reference puncture route is defined by the point 70A and the puncture angle φ1.

A sub guide line 80, which is an important element in the present embodiment, is a line segment representing the edge 82 of the sub area 72A. Here, an angle of tilt of the edge 82 is θ1. The sub guide line 80 is also tilted according to the deflection angle θ1. The sub guideline 80 is formed as a line shorter than the main guide line 78, and the main guide line 78 passes through a middle point of the sub guide line 80 in an orthogonal direction. The puncture guide 76 is desirably displayed in a form to not block observation of the puncture needle image and the tissue. In FIG. 3, the puncture guide 76 is expressed in an emphasized manner.

As described above, in order to clearly display the puncture needle image, it is desirable to hold the puncture needle within the sub area 72A. That is, it is desirable to determine an amount of insertion and the puncture angle such that the tip of the puncture needle does not reach the sub area 72B. However, the edge 82 is not clear, and it is difficult for the user to immediately recognize where the sub area 72A ends. On the contrary, according to the present embodiment, the reference puncture route is clearly identified on the screen by the main guide line 78, and the position of the edge 82 can be clearly identified by the sub guide line 80. Therefore, a shape or a size of the combining area 72A can be recognized prior to the freehand puncturing, and in particular, in a case where an arbitrary puncture angle is selected, a range in which the tip of the puncture needle can be inserted can be intuitively recognized.

Thus, it is possible to set the puncture angle φ1 beforehand while referring to the puncture guide 76 such that, for example, a target tissue image 64A is included in the sub area 72A, or a target coordinate 84, which is a center position of the target tissue image 64A, is included in the sub area 72A. Alternatively, the position and orientation of the probe may be changed beforehand so as to satisfy such a condition.

The puncture angle φ1 is designated by the user in the present embodiment. For example, the puncture angle φ1 is designated by turning a knob on the manipulation panel, or by touching the target coordinate 84 on the sub display. Alternatively, the coordinate may be automatically detected to automatically set the puncture angle φ1. Because there is a correspondence relationship as described above between the puncture angle φ1 and the deflection angle θ1, alternatively, the deflection angle θ1 may be designated in place of designation of the puncture angle φ1, and the puncture angle φ1 may consequently be determined indirectly.

In the present embodiment, prior to the freehand puncturing, a display image as shown in FIG. 3 is displayed, and the puncture angle φ1 is variably set while observing the display image. In this manner, the reference puncture route can be set at a suitable position and angle, and at the same time, a suitable deflection angle θ1 can be set. After it is confirmed that a suitable positional relationship is formed, the freehand puncturing is actually executed.

FIG. 4 shows a puncture guide 76 shown in FIG. 3. As described above, the sub guide line 80 is formed as a line segment extending along an edge of the combining area, and has an end 80A and an end 80B. The main guide line 78 passes through a middle point of these ends from an orthogonal direction. In the present embodiment, the sub guide line 80 has a recommended angle range 85. Specifically, a certain angle difference Δφ on an upper side and on a lower side with respect to the reference puncture route is shown by the ends 80A and 80B. By moving the puncture needle such that the puncture needle remains within the recommended angle range 85, it is possible to obtain a relatively clear image as the puncture needle image. When the puncture needle deviates from the recommended angle range 85, the possibility of the display of the puncture needle image becoming unclear is increased. The two ends 80A and 80B may be used as rough estimates during the insertion process. Here, Δφ is, for example, 5 degrees. Alternatively, a configuration may be employed in which the angle difference can be variably set by the user. In FIG. 4, two virtual lines 84A and 84B connecting the top right corner point of the display image and the two ends 80A and 80B are shown. These lines are not actually displayed, but alternatively, these lines may be displayed. A marker or the like representing a size of the recommended angle range 85 can be displayed partway on the main guide line 78.

The end 80A can be specified by subtracting Δφ from the puncture angle φ1, and the end 80B can be specified by adding Δφ to the puncture angle φ1. The coordinates of the two ends 80A and 80B of the sub guide line 80 are calculated by the graphic image formation unit described above.

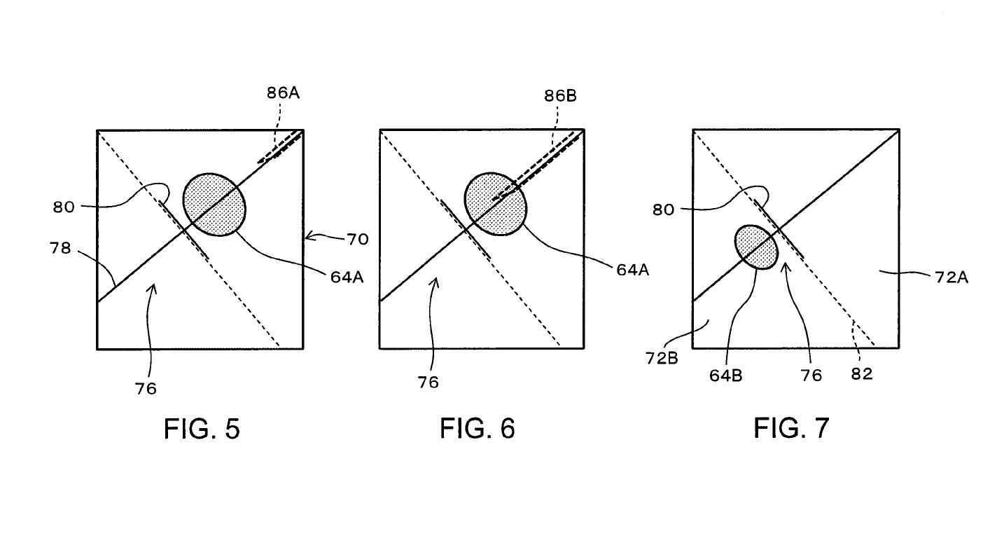

FIG. 5 shows a display image 70 immediately after the start of puncturing. The display image 70 includes the puncture guide 76 and also the target tissue image 64A. The main guide line 78 represents the reference puncture route, and the puncture needle image 86A is shown on the reference puncture route. The referenced puncture route is merely a rough estimate, and the puncture needle image 86A does not need to be placed on the reference puncture route. So long as the objective of the puncturing can be achieved, the puncture needle image 86A may deviate from the puncture route. FIG. 6 shows a state where the puncturing has proceeded further, and the tip of the puncture needle image 86B has reached the center of the target tissue image 64A.

As described, according to the puncture guide of the present embodiment, the limits of the puncture needle image formation can be easily recognized. Therefore, a problem as shown in FIG. 7 can be avoided. In FIG. 7, a target tissue image 64B is not in the sub area 72A, but exists in the sub area 72B. In such a case, if the puncture needle is moved closer to the target tissue, a problem may occur in which the puncture needle image disappears or becomes unclear around the point exceeding the edge 82. On the contrary, in the present embodiment, because the puncture guide 76 has the sub guide line 80 which represents the edge 82, that is, which represents the limitation of the clear image formation of the puncture needle, the problem of the puncturing being executed in the state as shown in FIG. 7 can be avoided in advance. Specifically, the user can execute the freehand puncturing after confirming that the point that will be reached by the puncture needle belongs to the subarea 72A before the freehand puncturing, for example, after confirming that the center of the target tissue image 64B exists within the sub area 72A.

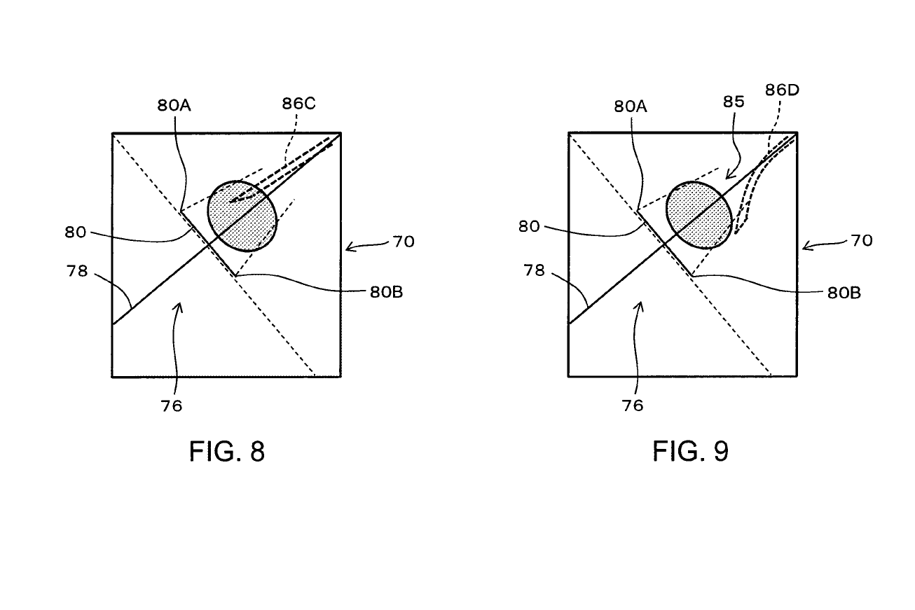

In the present embodiment, as described above, the sub guide line 80 also represents the recommended angle range. Therefore, when the puncture needle image 86C is within the angle range shown by the two ends 80A and 80B of the sub guide line 80 as shown in FIG. 8, an image of a certain clearness can be expected for the display image. On the other hand, when the tip or the like in the puncture needle image 86D deviates from the recommended angle range as shown in FIG. 9, such a situation can be easily recognized by comparing the puncture needle image 86 and the two ends 80A and 80B.

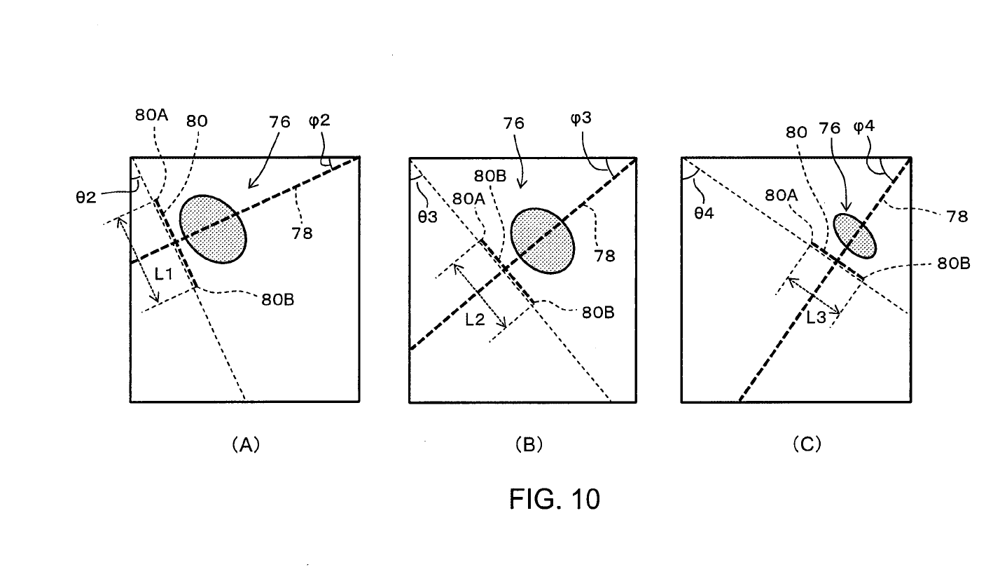

FIG. 10 shows a change of the puncture guide due to variation of the puncture angle. In (A), the puncture angle is φ2, in (B), the puncture angle is φ3, and in (C), the puncture angle is φ4. Here, the angles are in a relationship of φ2<φ3<φ4. Consequently, the deflection angles are in a relationship of θ2<θ3<θ4. As described above, the ends 80A and 80B of the sub guide line 80 represent the recommended angle range, and for example, are specified by adding and subtracting Δφ to and from the puncture angle. As can be understood with reference to (A) to (C), when the puncture angle is increased, the two ends 80A and 80B move in directions to become closer to each other. In other words, a line length L of the sub guide line 80 is reduced as the puncture angle is increased, with a relationship of L1>L2>L3. In other words, as the puncture angle, which is an angle between the reference puncture angle represented by the main guide line 78 and the horizontal line, is gradually reduced, the sub guide line 80 is slid and moved toward a depth side, that is, deeper side, on the main guide line 78, and at the same time, the line length of the sub guide line 80 is gradually increased. It is desirable to select a suitable puncture angle according to the position of the target tissue, and in this case, the puncture guide can be utilized.

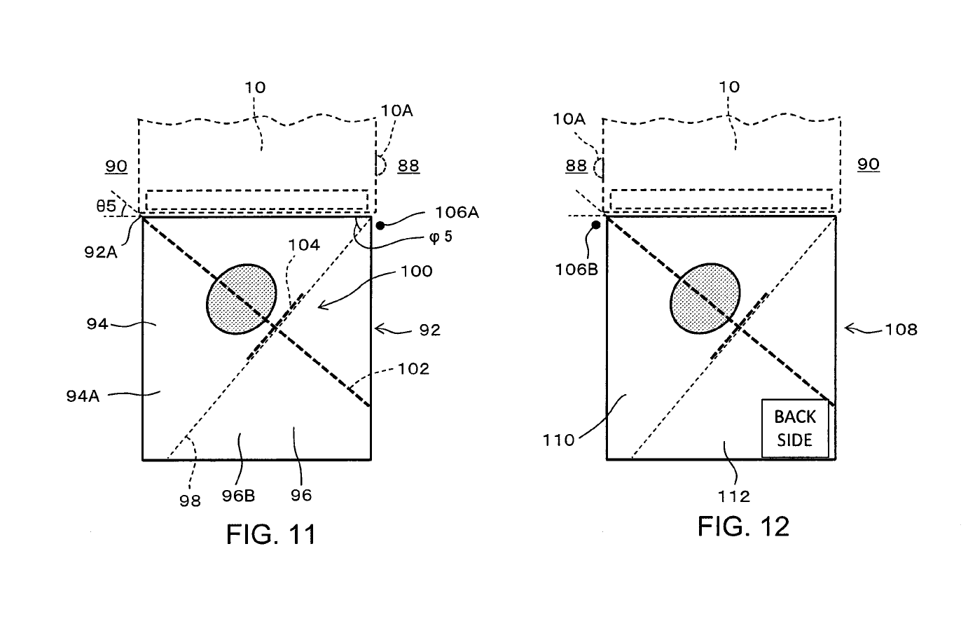

FIG. 11 shows a second display mode. With regard to the first display mode, the first display mode has already been explained with reference to FIG. 3.

In the probe 10, a side where the protrusion 10A is provided is a start end side 88. A side opposite thereto is a completion end side 90. In the present embodiment, the first display mode is selected when the puncture needle is inserted into the body at the start end side 88, and the second display mode is selected when the puncture needle is inserted into the body at the completion end side 90.

In the second display mode shown in FIG. 11, a first beam scan identical to the first beam scan in the first display mode is executed, and in the second beam scan, the ultrasound beam is electronically scanned with a negative deflection angle. The deflection angle is shown in FIG. 11 as θ5. A display image 92 is an image in which a tomographic image 94 and a graphic image 96 are combined, and the tomographic image 94 includes a sub area 94A and a sub area 96B. Reference numeral 98 represents an edge of the sub area 94A.

The graphic image 96 includes a puncture guide 100, which includes a main guide line 102 and a sub guide line 104. The main guide line 102 is tilted in a reverse direction from a top left corner point 92A in the display image 92 toward the bottom right. An angle of tilt of the main guide line 102, that is, the puncture angle, is φ5. In the present embodiment, the angle φ5 is defined as a positive angle. The sub guide line 104 is a line segment representing the edge 98, and is orthogonal to the main guide line 102.

As described above, in the second display mode, the ultrasound beam is scanned in a state of being tilted in the reverse direction, and the puncture guide 100 having a reverse tilt orientation is displayed. Therefore, depending on the dominant arm, or depending on a position of the target tissue or the like, the procedure and display mode may be selected. With such a configuration, the operability can be improved or accurate puncturing can be realized.

In FIG. 11, a marker 106A displayed near the display image 92 represents the start end side of the electronic scan. By observing the marker 106A, the relationship between the orientation of the probe 10 and the orientation of the image can be intuitively recognized.

FIG. 12 shows an inverted display mode. When the probe 10 is in an inverted state in the forward-rear direction for any reason, the start end side of the probe 10 in the actual space and the start end side on the screen do not match. In such a case, an inverted display may be commanded, to execute an inverted display mode, to invert a display image 108 in the left-right direction. That is, a tomographic image 110 is displayed in an inverted state, and a graphic image 112 is also displayed in an inverted state. In other words, back sides of these images are displayed. In the display image 108, a marker 106B is displayed on a side opposite to that in the normal display. In FIG. 12, the first display mode is selected and the inverted display mode is selected. Alternatively, the inverted display mode may be selected in the second display mode.

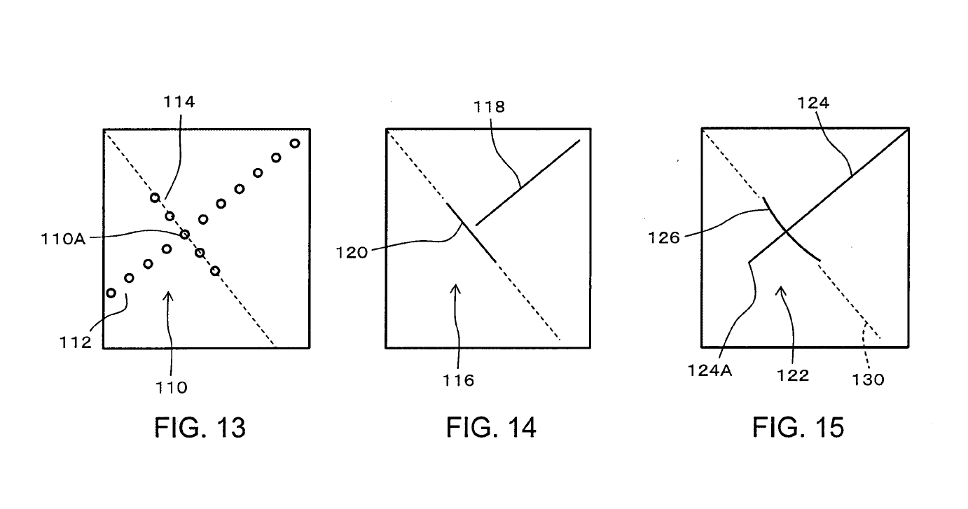

Next, another example of the puncture guide will be described. FIG. 13 shows a second example of the puncture guide. A puncture guide 110 comprises a main guide line 112 and a sub guide line 114. The lines 112 and 114 are each expressed with a plurality of points. Reference numeral 110A shows a point corresponding to a crossing position of the two lines. Alternatively, the display of the point 110A may be omitted. According to the puncture guide 110 as shown in FIG. 13, an advantage can be obtained in that the lines do not block the views during observation of the tissue and the puncture needle image too much. The points may be formed in a circular shape with the inside filled, or in a ring shape with the inside not filled.

FIG. 14 shows a third example of the puncture guide. A puncture guide 116 has a T shape. Specifically, a main guide line 118 does not penetrate through a sub guide line 120, and the main guide line 118 remains within the overlap sub area. With such a puncture guide 116 also, the above-described operational advantages can be obtained. In particular, with the T-shaped guide, an advantage can be obtained in that an insertion limit can be more intuitively recognized.

FIG. 15 shows a fourth example of the puncture guide. A puncture guide 122 comprises a main guide line 124 and a sub guide line 126, and an end edge 124A of the main guide line 124 does not reach the edge of the display image, and remains in the partway. The sub guide line 126 is formed in an arc form centered at a point at atop right corner. With such a curved form also, the edge, that is, the boundary 130, can be recognized. In addition, with the arc-shaped sub guide line 126, an advantage can be obtained in that the recommended angle range can be more intuitively recognized.

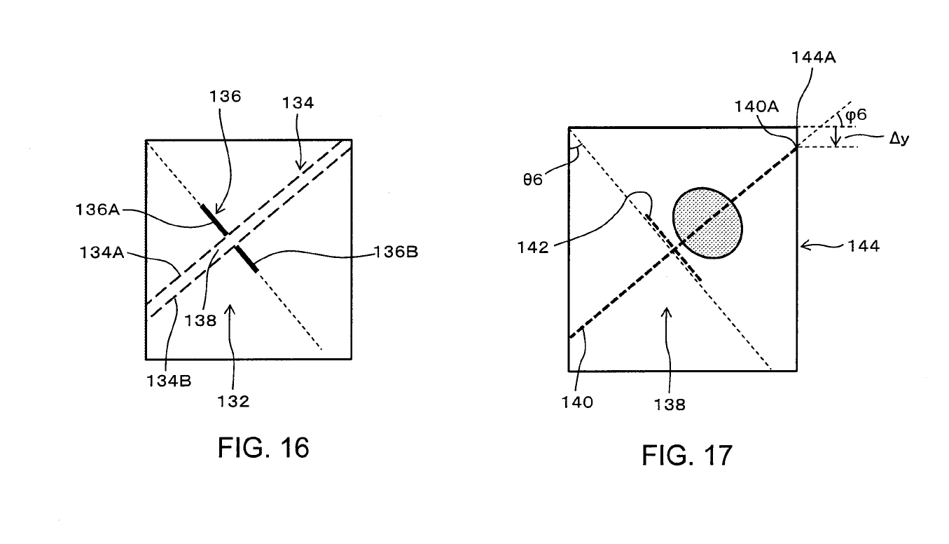

FIG. 16 shows a fifth example of the puncture guide. A puncture guide 132 comprises a main guide line 134 and a sub guide line 136. The main guide line 134 more specifically includes two lines 134A and 134B which extend parallel to each other, and the lines are drawn on both sides of the reference insertion route. With such a configuration, a problem of the main guide line 134 being drawn on the puncture needle image and thereby blocking the observation of the puncture needle image can be reduced. The sub guide line 136 is divided, that is, the sub guide line 136 includes line segments 136A and 136B. A gap exists between the line segments 136A and 136B, which corresponds to a distance between the two lines 134A and 134B.

Alternatively, only one line of the two lines 134A and 134B shown in FIG. 16 may be displayed, and set as the main guide line. That is, the main guide line may be formed not as a line drawn on the reference puncture route, but as a line parallel to this line and offset by a predetermined distance therefrom. Similarly, the sub guide line may be displayed at a position offset from the edge by a certain distance.

FIG. 17 shows a sixth example of the puncture guide. A puncture guide 138 has a form similar to that of the puncture guide shown in FIG. 3, but differs therefrom in the position in the y direction. Specifically, a main guide line 140 extends from a point 140A shifted downward from a point 144A at the top right corner of a display image 144 by a certain distance Δγ. For example, depending on the shape of the probe, it may be difficult to puncture while passing the point 144A, and in such a case, the display form as shown in FIG. 17 may be employed. When this display form is to be employed, a puncture angle θ and the distance Δγ may be designated in the formation of the puncture guide 138. Here, Δγ may be configured to be variably set by the user or to be automatically determined according to, for example, the type of the probe.

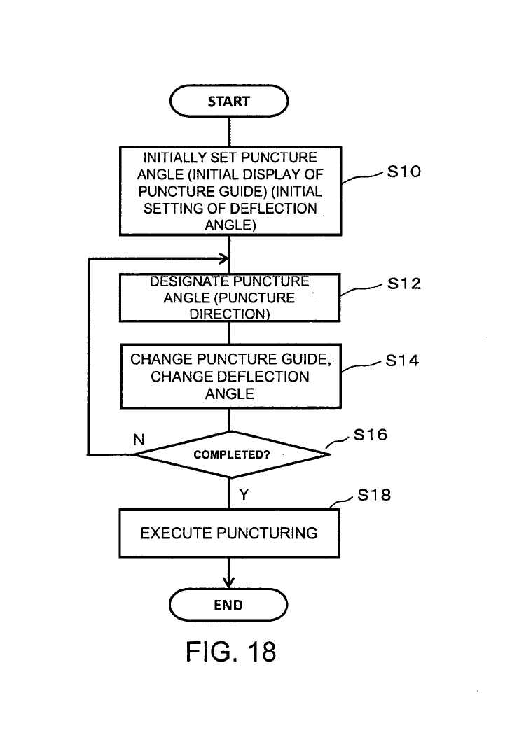

FIG. 18 shows an example operation of the ultrasound diagnostic apparatus of FIG. 1. In S10, an initial setting of the puncture angle is executed. For example, an initial value is given as the puncture angle. The deflection angle is initially set based on the puncture angle thus designated, and the puncture guide is displayed in an initial form. With the initial setting, the first beam scan and the second beam scan are alternately executed, the tomographic image is displayed on the display screen, and the puncture guide as described above is also displayed. Alternatively, the tomographic image may be displayed from a time prior to the execution of S10.

In S12, the user manipulates on a knob or the like, to designate the puncture angle. When a new puncture angle is set in S12, the deflection angle is correspondingly changed in S14, and the display form of the puncture guide is also changed.

The processes of S12 and S14 are repeated until the completion of designation of the puncture angle is judged in S16. With such a process, the user can set the reference puncture direction to a suitable direction in relation to the target tissue while referring to the puncture guide. That is, the user can set a suitable puncture angle. In S18, the free-hand puncturing is actually executed after the above-described setting is completed.

In the present embodiment, the puncture angle can be variably set while viewing the tomographic image which is displayed in real time. In this case, because the deflection angle is changed in real time corresponding to the puncture angle and the display form of the puncture guide is also changed accordingly, a suitable puncture angle can be designated quickly. In addition, an outer edge of a range in which the puncture needle can be clearly displayed can be checked before the puncturing.

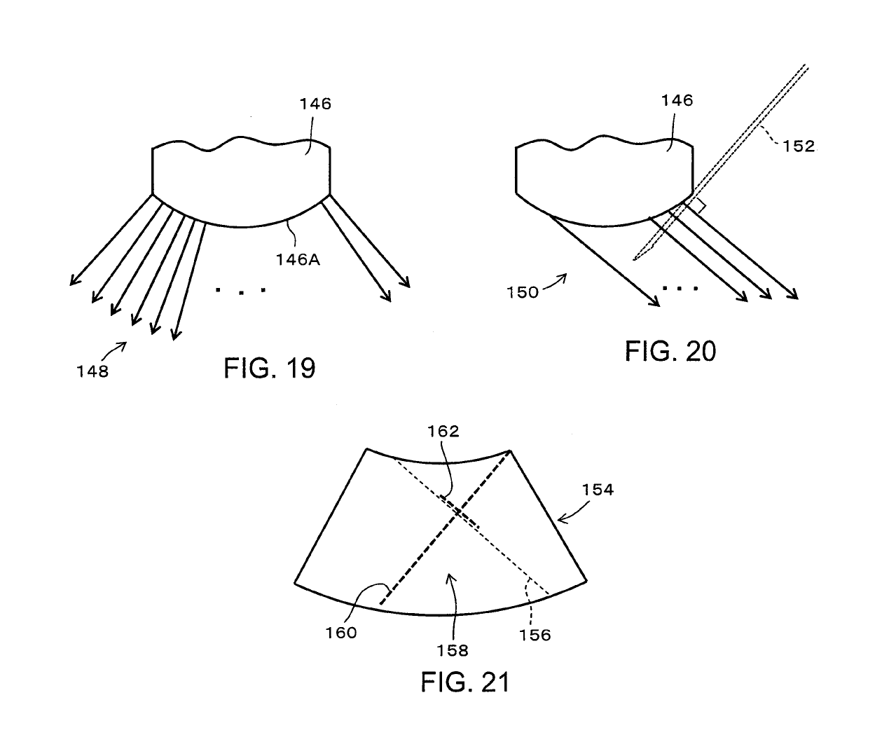

FIGS. 19-21 show examples of a puncture guide display in a convex probe. In FIG. 19, a convex probe 146 has a curved transmission and reception plane 146A having an arc shape. In the convex probe 146, when the beam scan is executed under a condition of the deflection angle of 0 degrees, a first scan plane 148 is formed. On the other hand, when the ultrasound beams are sequentially formed such that predetermined deflection angles are formed in the living body as shown in FIG. 20, a second scan plane 150 is formed. In this case, the formation condition of each ultrasound beam is desirably determined such that each ultrasound beam is orthogonal to the puncture needle 152, as described above.

FIG. 21 shows a display image 154. The display image 154 is an image in which a tomographic image and a graphic image are combined. An ultrasound image is an image formed based on combined frame data in which first frame data corresponding to the first scan plane and second frame data corresponding to the second scan plane are combined. Therefore, similar to the case in which the linear probe is used, an edge of the overlap sub area would be generated. This edge is shown with reference numeral 156. A puncture guide line 158 comprises a main guide line 160 and a sub guide line 162 orthogonal to the main guide line 160. The sub guide line 162 represents the reference puncture route corresponding to the puncture angle designated by the user, and the sub guide line 162 is a line representing the edge 156. In this manner, in the case where the convex probe is used also, the puncture guide may be utilized.

The lines forming the puncture guide described above can be displayed in an arbitrary form, and with an arbitrary hue. The puncture guide is desirably displayed in a form that does not block the observation of the puncture needle image and the tissue.

1-14. (canceled)

15. An ultrasound diagnostic apparatus comprising:
a deflection angle determination unit that determines a beam deflection angle corresponding to a planned puncture angle which is an angle from a horizontal line;
a scan controller that controls a first beam scan for forming a tissue image, and a second beam scan for forming a puncture needle image according to the beam deflection angle;
a graphic image formation unit that forms a graphic image having a puncture guide;
a combining unit that combines first frame data obtained by the first beam scan and second frame data obtained by the second beam scan, to produce combined frame data; and
a display processor that combines an ultrasound image based on the combined frame data and the graphic image, to produce a display image, wherein
the puncture guide includes:
a main guide line that represents a reference puncture route determined from the planned puncture angle; and
a sub guide line that intersects the main guide line, that is shorter than the main guide line, and that represents an edge of a second beam scan area formed by the second beam scan, and a position of the sub guide line is moved toward a deeper position along the main guide line as the planned puncture angle is reduced, the reference puncture route becomes closer to a horizontal line, and the beam deflection angle is reduced.

16. The ultrasound diagnostic apparatus according to claim 15, wherein
the sub guide line is orthogonal to the main guide line, and
a combination of the main guide line and the sub guide line forms a cross shape or a T shape.

17. The ultrasound diagnostic apparatus according to claim 15, wherein
coordinates of both ends of the sub guide line represent a lower limit and an upper limit of a recommended puncture angle range determined with reference to the reference puncture route.

18. The ultrasound diagnostic apparatus according to claim 15, wherein
a position of the sub guide line is moved toward a deeper position along the main guide line, and a line length of the sub guide line is increased as the planned puncture angle is reduced, the reference puncture route becomes closer to a horizontal line, and the beam deflection angle is reduced.

19. The ultrasound diagnostic apparatus according to claim 18, wherein
the upper limit and the lower limit of the recommended puncture angle range are determined by an addition and a subtraction of a predetermined angle to and from the planned puncture angle.

20. The ultrasound diagnostic apparatus according to claim 15, further comprising an emphasis processor that applies an emphasis processing to emphasize an image of the puncture needle on the second frame data, wherein the combining unit combines the second frame data after the emphasis processing with the first frame data.

21. The ultrasound diagnostic apparatus according to claim 20, wherein the emphasis processing includes an edge emphasis processing.

22. The ultrasound diagnostic apparatus according to claim 15, further comprising:
a probe that has a transmission and reception end in which an array transducer formed from a plurality of transducer elements that transmit and receive ultrasound is built in, and that is in contact with a surface of a living body; and
a mode selector that selects a display mode from among a first display mode adapted for one side procedure in which puncturing is executed on one side of the transmission and reception end, and a second display mode adapted for an other side procedure in which the puncturing is executed on the other side of the transmission and reception end,
wherein when the first display mode is selected, the scan controller sets a positive angle as the beam deflection angle and a forward tilted puncture guide having a main guide line tilted from an upper part on the one side toward a lower part on the other side is displayed as the puncture guide, and
wherein when the second display mode is selected, the scan controller sets a negative angle as the beam deflection angle, and a reverse tilted puncture guide having a main guide line tilted from an upper part on the other side toward a lower part on the one side is displayed as the puncture guide.

23. The ultrasound diagnostic apparatus according to claim 15, wherein the display processor inverts the ultrasound image and the graphic image in the left-right direction when an inversion command is input.

24. The ultrasound diagnostic apparatus according to claim 15, further comprising an input unit that allows variation of the planned puncture angle, wherein
a real-time tomographic image is displayed as the ultrasound image, and
a beam deflection angle in the second beam scan and a display form of the puncture guide are updated in real time according to a change of the planned puncture angle.

25. The ultrasound diagnostic apparatus according to claim 15, wherein the ultrasound diagnostic apparatus is an apparatus used when freehand puncturing is executed without the use of a puncture adapter that mechanically guides a puncture needle.
